(19) United States
(12) Patent Application Publication
(10) Pub. No.: US 2026/0192086 A1
(43) Pub. Date: Jul. 9, 2026

(54) Urinary Catheter and Bag Packaging Systems

(71) Applicant: C. R. Bard, Inc., Franklin Lakes, NJ (US)

(72) Inventors: Ronald N. Legaspi, Alpharetta, GA (US); Pooja Bhuvanesh Kulkarni, Pune (IN); Sudev Geetha Surendran, Kozhikode District (IN); James David Hughett, Sr., Monroe, GA (US)

(21) Appl. No.: 19/131,892

(22) PCT Filed: Nov. 21, 2022
(86) PCT No.: PCT/US2022/050646

§ 371 Date: May 21, 2025

(51) Int. Cl. A61M 25/00; A61M 39/10

(52) U.S. Cl. CPC A61M 25/002; A61M 25/0068; A61M 25/0097; A61M 39/10; A61M 2025/0006; A61M 2039/1027; A61M 2202/0014; A61M 2202/0496; A61M 2205/58; A61M 2209/06; A61M 2210/1085; A61M 2210/1089

(57) ABSTRACT

Fluid collection systems including an outer packaging that contains a catheter assembly during transport and storage and can convert to a fluid collection container during use. The catheter can be stored within the packaging to protect the device, and any lubricious coating disposed thereon, during transport and storage. During use, the catheter can be removed from the packaging and a proximal end of the catheter can be coupled to an inlet of the packaging and transition the packaging to a fluid collection container. The catheter can be inserted into a body of a patient to drain a fluid therefrom. The fluid can then be collected within the fluid collection container. Advantageously, the catheter can be easily detached from the collection container to allow for discrete emptying and/or disposal of the collection container.

### SUMMARY

Briefly summarized, embodiments of the present invention are directed to fluid collection systems including an outer packaging that contains a catheter assembly during transport and storage and can convert to a fluid collection container during use. The catheter can be stored within the packaging to protect the device, and any lubricious coating disposed thereon, during transport and storage. During use, the catheter can be removed from the packaging and a proximal end of the catheter can be coupled to an inlet of the packaging and transition the packaging to a fluid collection container. The catheter can be inserted into a body of a patient to drain a fluid therefrom. The fluid can then be collected within the fluid collection container. Advantageously, the catheter can be easily detached from the collection container to allow for discrete emptying and/or disposal of the collection container.

Disclosed herein is a fluid collection system transitionable between a storage state and a drainage state the system including, an outer packaging defining an interior cavity and including an inlet in fluid communication with the interior cavity, a catheter assembly having a distal portion disposed within the interior cavity in the storage state, the catheter assembly including, a catheter body defining a catheter lumen and including an eyelet disposed at the distal tip to provide fluid communication with the catheter lumen, a hub coupled to a proximal end of the catheter body and defining an outlet in fluid communication with the catheter lumen, the hub engaged with the inlet in the storage state, and a collapsible sheath extending distally from the hub and extending co-axially over the catheter body.

In some embodiments, the hub, in the storage state, is releasably secured within the inlet in an interference-fit, press-fit, snap-fit, or threaded engagement.

In some embodiments, a proximal end of the hub extends from the inlet when the fluid collection system is in the storage state.

In some embodiments, the fluid collection system further includes an abutment disposed distally of the outlet and configured to abut against a portion of the inlet to prevent the outlet from engaging the inlet in the storage state.

In some embodiments, the catheter assembly is configured to be removed from the interior cavity and the outlet disposed at a proximal end of the hub is coupled to the inlet to transition the system to the drainage state.

In some embodiments, the hub outlet engages the inlet in one of an interference-fit, press-fit, snap-fit, threaded, bayonet, or luer lock engagement in the drainage state.

In some embodiments, the fluid collection system further includes a loop coupled to a bottom edge of the outer packaging and configured to engage the inlet in the storage state.

In some embodiments, the catheter assembly is configured to drain a fluid from a bladder of a patient and the outer packaging is configured to collect the fluid within the interior cavity.

In some embodiments, the fluid collection system further includes a cap configured to releasably engage the inlet and cover one or both of the inlet and the hub outlet.

In some embodiments, the cap is configured to form a fluid tight seal with the inlet when engaged therewith.

In some embodiments, the fluid collection system further includes a handle coupled to a distal end of the sheath and slidably engaged with the catheter body, the handle configured to guide the distal tip of the catheter body into a urethra and collapse the sheath as the distal tip is advanced distally.

In some embodiments, a distal tip of the handle is configured to engage the outlet of the hub to form the sheath into a loop and contain the catheter body therein.

Also disclosed is a method of use for a fluid drainage system including, providing the fluid drainage system in a storage state, having an outer packaging defining an interior cavity and including an inlet, a catheter assembly, a distal portion thereof disposed within the interior cavity, the catheter assembly including, a catheter body defining a catheter lumen, a hub coupled to a proximal end of the catheter body and defining an outlet in fluid communication with the catheter lumen, and a collapsible sheath extending distally from the hub and extending co-axially over the catheter body, grasping a proximal end of the hub, a distal portion of the hub releasably secured within the inlet, removing the catheter assembly from the interior cavity, and coupling the proximal end of the hub with the inlet to place the catheter lumen in fluid communication with the interior cavity and transition the fluid drainage system to the drainage state.

In some embodiments, the hub, in the storage state, is releasably secured within the inlet in an interference-fit, press-fit, snap-fit, or threaded engagement.

In some embodiments, the proximal end of the hub extends from the inlet when the fluid drainage system is in the storage state.

In some embodiments, the method further includes abutting an abutment, disposed distally of the proximal end of the hub, against a portion of the inlet to prevent the proximal end of the hub from engaging the inlet in the storage state.

In some embodiments, the method further includes engaging the proximal end of the hub with the inlet in one of an interference-fit, press-fit, snap-fit, threaded, bayonet, or luer lock engagement, in the drainage state.

In some embodiments, the method further includes coupling a loop, which is attached to a bottom edge of the outer packaging opposite the inlet, with the inlet in the storage state to secure the outer packaging in a folded state.

In some embodiments, the method further includes draining a fluid from the catheter body into the interior cavity of the outer packaging.

In some embodiments, the method further includes releasably engaging a cap with the inlet to cover one or both of the inlet and the proximal end of the hub.

In some embodiments, the method further includes forming a fluid tight seal between the cap and the inlet when the cap is engaged therewith.

In some embodiments, the method further includes urging a handle proximally towards the hub, to collapse the sheath between the handle and the hub and to advance a distal tip of the catheter body distally of the handle, the handle coupled to a distal end of the sheath.

In some embodiments, the method further includes sliding the handle distally from the hub to withdraw a portion of the catheter body through the handle and into the sheath and coupling a distal tip of the handle with a proximal end of the hub to form the sheath into a loop and contain the catheter body therein.

Also disclosed is a method of manufacturing a fluid collection system transitionable between a storage state and a drainage state, the method including, forming an outer packaging defining an interior cavity and including an inlet in fluid communication with the interior cavity, forming a catheter assembly having a catheter body defining a catheter lumen and including an eyelet disposed at the distal tip to provide fluid communication with the catheter lumen, a hub coupled to a proximal end of the catheter body and defining an outlet in fluid communication with the catheter lumen, and a collapsible sheath extending distally from the hub and extending co-axially over the catheter body, placing a distal portion of the catheter assembly within the interior cavity, engaging the hub with the inlet such that a proximal end of the hub extends from the inlet, and enclosing one or both of the inlet and the proximal end of the hub with a cap.

In some embodiments, forming the outer packaging includes joining a first sheet of flexible material with a second sheet of flexible material along an edge thereof using adhesive, bonding, welding, or ultrasonic welding to define the interior cavity.

In some embodiments, the inlet is formed as a separate structure and secured between the first sheet of flexible material and the second sheet of flexible material to provide fluid communication with the interior cavity.

In some embodiments, the hub is releasably secured within the inlet in an interference-fit, press-fit, snap-fit, or threaded engagement.

In some embodiments, the method further includes abutting an abutment, disposed distally of the proximal end of the hub, against a portion of the inlet to prevent the proximal end of the hub from entering the inlet.

In some embodiments, the method further includes engaging a loop, coupled to a bottom edge of the outer packaging, with the inlet that is coupled to a top edge of the outer packaging, to secure the outer packaging in a folded state.

In some embodiments, the method further includes forming a fluid tight seal between the cap and the inlet to secure the catheter assembly within a sterile environment defined by the cap and the outer packaging.

Also disclosed is a method of transitioning a fluid collection system from a storage state to a drainage state including, disengaging a cap from an inlet of an outer packaging, disengaging a hub of a catheter assembly from the inlet, removing the catheter assembly from an interior cavity defined by the outer packaging, and coupling a proximal end of the hub to the inlet to place a lumen of the catheter assembly in fluid communication with the interior cavity.

In some embodiments, the method further includes disengaging a loop from the inlet, the loop coupled to a bottom edge of the outer packaging, opposite from the inlet disposed at a top edge of the outer packaging, and unfolding the outer packaging.

In some embodiments, an outlet of the hub extends from the inlet in the storage state, and is disposed within the inlet in the drainage state.

In some embodiments, the outer packaging and the cap co-ordinate to maintain the catheter assembly within a sterile environment when the fluid collection system is in the storage state.

In some embodiments, the catheter assembly, includes a catheter body defining the lumen, the hub coupled to a proximal end of the catheter body, the outlet of the hub in fluid communication with the lumen, and a collapsible sheath extending co-axially over the catheter body.

Also disclosed is a method of storing a fluid collection system including, draining a fluid from a lumen of a catheter body of a catheter assembly into an interior cavity defined by an outer packaging, disengaging an outlet of a hub of the catheter assembly from an inlet of the outer packaging, and coupling a cap to the inlet to form a fluid tight seal therebetween.

In some embodiments, the method further includes sliding the catheter body distally through a handle to enclose the catheter body within a sheath, the sheath extending coaxially over the catheter body between the hub and the handle.

In some embodiments, the method further includes coupling a distal end of the handle to a proximal end of the hub to form a loop and secure the catheter body within the sheath.

In some embodiments, the method further includes disengaging the cap from the inlet and emptying the fluid from the interior cavity of the outer packaging.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of an outer packaging of a fluid collection system, in accordance with embodiments disclosed herein.

FIG. 1B shows a perspective view of a catheter assembly of a fluid collection system, in accordance with embodiments disclosed herein.

FIG. 2A shows close up detail of a catheter assembly engaged with an inlet of the outer packaging in a storage state, in accordance with embodiments disclosed herein.

FIG. 2B shows close up detail of a catheter assembly being removed from the outer packaging, in accordance with embodiments disclosed herein.

FIG. 2C shows close up detail of a proximal end of a catheter assembly being coupled with the inlet of the outer packaging in a drainage state, in accordance with embodiments disclosed herein.

FIGS. 3A-3D show a method of transitioning the fluid drainage system from a storage state to a drainage state, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention, and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

To assist in the description of embodiments described herein a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

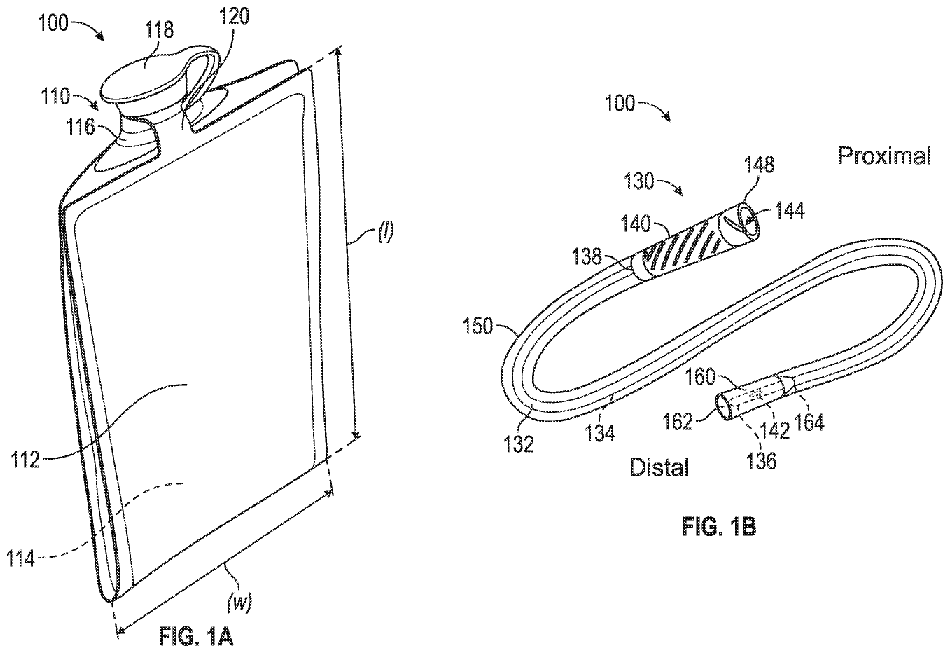

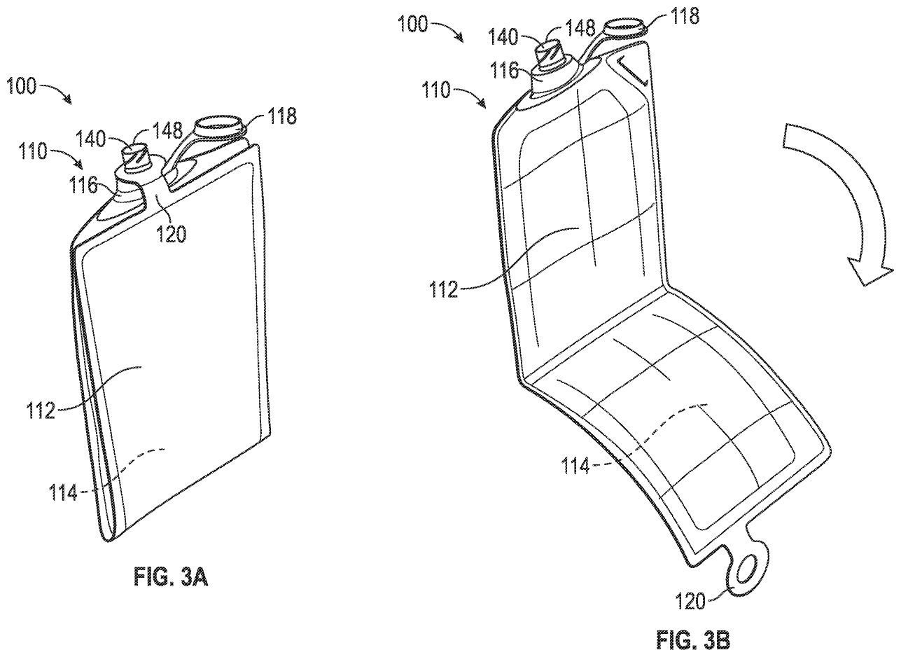

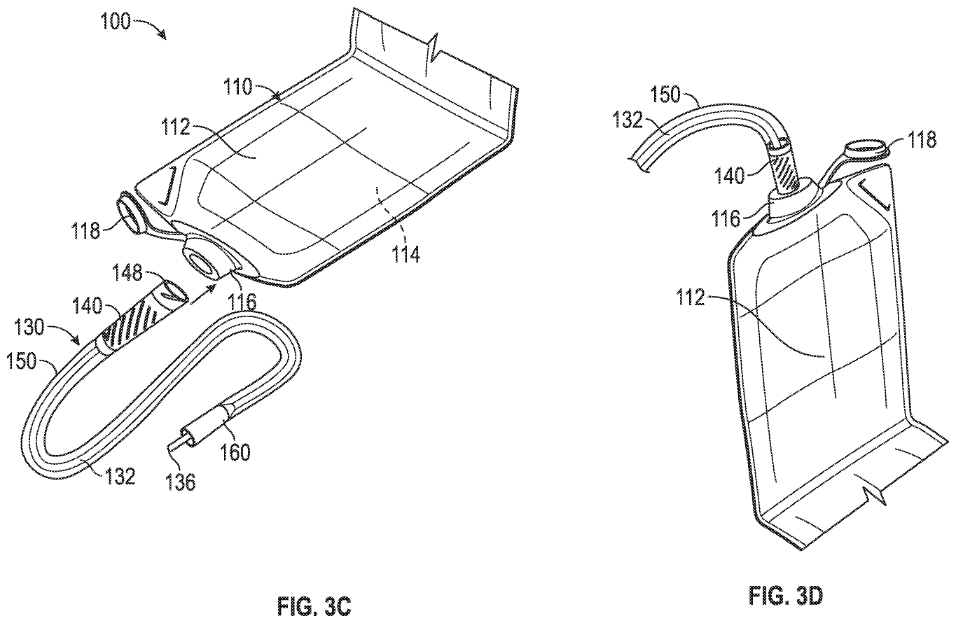

FIGS. 1A-1B show an exemplary fluid collection system (“system”) 100 configured to drain a fluid from a body of a patient. The system 100 can generally include an outer packaging 110 (FIG. 1A) and a catheter assembly 130 (FIG. 1B) and can be transitionable between a storage state (FIG. 3A) and a drainage state (FIG. 3D). Exemplary fluids to be drained by the drainage system 100 can include blood, urine, peritoneal fluid, pus, or the like. In an embodiment, the system 100 can be configured to drain urine from a bladder of a patient.

As shown in FIG. 1A, the outer packaging 110 can generally include a container body 112 defining a length (l) and a width (w). The body 112 can be formed of a flexible material or a rigid material, such as a plastic, polymer, thermoplastic, composite, or the like. As shown, the body 112 is formed of two flexible sheets of material joined along the edges using adhesive, bonding, welding, ultrasonic welding, or the like to define an interior cavity 114. The outer packaging 110 can further include an inlet 116 providing fluid communication with the interior cavity 114 of the body 112.

In an embodiment, the inlet 116 can include a cap 118 configured to engage the inlet 116 and prevent fluid communication therethrough. Optionally, the cap 118 can be tethered to the inlet 116 or to the body 112. The cap 118 can engage the inlet 116 in a snap-fit, interference fit, press-fit, a bayonet-fit, luer-lock, threadable engagement, or similar suitable engagement. In an embodiment, one or both of the inlet 116 and the cap 118 can be formed of the same material as the body 112. In an embodiment, one or both of the inlet 116 and the cap 118 can be formed of a different material from the body 112, displaying different mechanical properties. In an embodiment, one or both of the inlet 116 and the cap 118 can be formed of a relatively rigid material, relative to the body 112.

In an embodiment, the outer packaging 110 can further include a loop 120, or similar structure coupled to, or formed integrally with, an edge of the body 112. As shown for example in FIG. 3B, the loop 120 can be coupled to a bottom edge of the body 112 opposite from the inlet 116, which is coupled to a top edge. The loop 120 can engage one or both of the inlet 116 and the cap 118 to secure the outer packaging in a folded configuration (FIGS. 1A, 3A). Advantageously, the folded configuration can maintain the system 110 in a more compact profile during transport and storage.

As shown in FIG. 1B, a catheter assembly 130 of the system 100 can include a catheter body 132 extending longitudinally, and defining a lumen 134. The catheter body 132 can extend between a distal end 136 and a proximal end 138. The distal end 136 can include an eyelet 142 providing fluid communication with the lumen 134. In an embodiment, the distal tip 136 can be configured to be inserted into a urethra to access a bladder of a patient and drain a fluid therefrom. A proximal end 138 of the catheter body 132 can be supported by a hub 140 that defines a hub lumen 144. The hub lumen 144 is in fluid communication with the catheter body lumen 134 and can extend to a hub outlet at a proximal end 148. In an embodiment, the catheter body 132 can include a lubricious coating, anti-bacterial coating, or similar coating or combinations thereof, disposed on at least a portion of an outer surface thereof.

The catheter assembly 130 can further include a collapsible sheath 150 formed of a thin, gas impervious film, or similar material, and extending annularly about the catheter body 132. A proximal end of the sheath 150 can be coupled to a distal end of the hub 140 and can extend co-axially over the catheter body 132 to a distal end 136 thereof. Advantageously, the sheath 150 can protect the catheter body 132 during transport and storage. The sheath 150 can prevent direct touch contact which can contaminate portions of the catheter body 132 that are to be inserted into a body of the patient. Further, the sheath 150 can protect the coating and prevent the coating from drying out. In use, the sheath 150 can be configured to collapse as the catheter body 132 is inserted into the patient.

In an embodiment, the catheter assembly 130 can include a handle 160 disposed distally and defining a channel 164. A distal end of the sheath 150 can be coupled to a proximal end of the handle 160. In an embodiment, one or both of the hub 140 and the handle 160 can be formed of plastic, polymer, elastomer, rubber, silicone rubber, or the like. In an embodiment, one or both of the hub 140 and the handle 160 can include a gripping surface disposed on an outer surface thereof, including ridges, grooves, or similar textured surface or additional materials configured to provide improved grasping for a user. Further, the gripping surface(s) of the hub 140 and/or handle 160 can indicate to a user an appropriate place to grasp and manipulate the catheter assembly 130.

In an embodiment, the handle channel 164 can include a valve 162 or frangible seal extending across the channel 164 and configured to control a fluid flow through the channel 164 during transport and storage. Advantageously, one or more of the sheath 150, handle 160 and valve 162 can co-operate to contain the catheter body 132, and any coating disposed thereon, within a sealed and/or sterile environment, preventing the catheter body 132 and coating from drying out. Further, the valve 162 can mitigate exposure of the catheter body 132 to pathogens or contaminants. During use, a distal tip 136 of the catheter body 132 can be urged through the valve 162 which can separate and allow the distal tip 136 to extend distally of the handle 160. In an embodiment, an inner diameter of the channel 164 can be equal to, or slightly larger than an outer diameter of the catheter body 132. In an embodiment, the catheter body 132 can be slidably engaged with the channel 164.

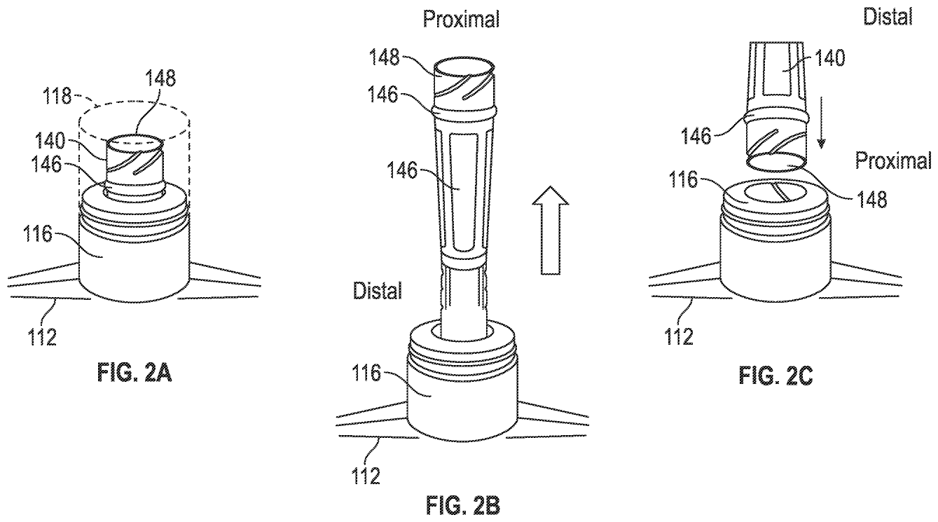

FIGS. 2A-2C show further details of the hub 140 and inlet 116 of the system 100. When the system 100 is in a storage state, a distal portion of the catheter assembly 130 can be disposed within the cavity 114 of the body 112, with a portion of the outer surface of the hub 140 engaging an inner surface of the inlet 116 in an interference fit, press-fit, snap-fit, bayonet-fit, luer-lock, or threadable engagement. In an embodiment, a portion of an outer surface of the hub 140 can define a tapered outer profile to facilitate engaging the inlet 116 in one of an interference fit, press-fit, or luer-lock engagement.

FIG. 2A shows close up detail of the hub 140 engaged with the inlet 116, with a distal portion of the catheter assembly 130 disposed within the cavity 114 of the body 112. In an embodiment, the inlet 116 can be formed of a relatively rigid material such as a plastic, polymer, thermoplastic, composite, or the like. In an embodiment, the hub 140 can be formed of a relatively compliant material, relative to the inlet 116, such as a plastic, polymer, elastomer, rubber, silicone rubber, composite, or the like.

In an embodiment, the hub 140 can include an abutment 146, such as an annular ring, or similar structure configured to abut against a surface of the inlet 116 and prevent a proximal end 148 of the hub 140 from entering the inlet 116. As such, as shown in FIG. 2A, a proximal portion of the hub 140, disposed proximally of the abutment 146 can extend from the inlet 116 when the hub 140 is engaged with the inlet 116 in the storage state. In an embodiment, the hub 140 can engage the inlet 116 creating a seal therebetween and preventing a fluid flow between the outer surface of the hub 140 and an inner surface of the inlet 116. Advantageously, seal engagement between the hub 140 and the inlet 116 can mitigate the introduction of pathogens or contaminants to the interior cavity 114 while the system 100 is in the storage state.

In an embodiment, the cap 118 can engage the inlet 118 in one of an interference fit, press-fit, snap-fit, bayonet, luer-lock, or threadable engagement and can cover one or both of the inlet 116 and the proximal end 148 of the hub 140 extending therefrom. Advantageously, the cap 118 can provide a seal preventing contaminants from contacting the catheter assembly 130 or the interior cavity 114 of the packaging 110 while the system 100 is in the storage state.

In use, as shown in FIGS. 2A-2C, a user can disengage the cap 118 and grasp a proximal portion of the hub 140 to disengage the hub 140 from the inlet 116 (FIG. 2B). The user can then remove the catheter assembly 130 from the interior cavity 114. As shown in FIG. 2C, the user can then invert the catheter assembly 110 and engage a proximal end 148 of the hub 140 with an inner surface of the inlet 116 to transition the system 110 from the storage state to a drainage state. The proximal end 148 of the hub 140 can engage the inlet 116 in one of an interference fit, press-fit, snap-fit, bayonet, luer-lock, or threadable engagement.

FIGS. 3A-3D show an exemplary method of use of a fluid drainage system 100. As shown in FIG. 3A, the system 100 can be provided in folded, storage state with the catheter assembly 130 disposed within the interior cavity 114, the hub 140 engaged with the inlet 116 and covered by the cap 118. Optionally, a loop 120 can engage the inlet 116 to maintain the body 112 in a folded storage state. Advantageously, in the storage state, the entire catheter assembly 130 is secured within a sterile environment of the interior cavity 114. As such, the body 112 can serve as an outer packaging 110 to the system 100. This can prevent the catheter assembly 130, especially any lubricious coating, from drying out during transport or storage. Further, the catheter assembly 130 is protected from any dust or contaminants during transport or storage. Further, the loop structure 120 can maintain the outer packaging 110 in a folded state reducing an overall profile of the system 100 in the storage state.

As shown in FIGS. 3A-3B, a user can disengage the cap 118 from the inlet 118 exposing a proximal end 148 of the hub 140. The user can then disengage the loop 120 from the inlet 116 and unfold the body 112 (FIG. 3B). As shown in FIGS. 2B and 3C, the user can grasp the proximal end 148 of the hub 140 extending from the inlet 116 and disengage the hub 140 from the inlet 116. The catheter assembly 130 can then be removed entirely from the interior cavity 114 of the outer packaging 110. The user can then invert the catheter assembly 110 and can engage the outlet at a proximal end 148 of the hub 140 with the inlet 116. In an embodiment, and outer surface of the proximal end of the hub 140 can engage an inner surface of the inlet 116, e.g. in a threadable engagement, or the like.

In an embodiment, the user can then place the catheter body 132 by grasping the handle 160 and aligning the distal tip 136 of the catheter body 132 with a target location, e.g. a urethra. The user can then slide the handle 160 proximally relative to the distal tip 136. The sheath 150 can collapse to allow the distal tip 136 to slide distally of the handle 160, breaking the seal or traversing the valve 162 disposed within the channel 164, and entering the urethra. In an embodiment, the user can support the hub 140 and urge the hub 140 distally to urge the catheter body 132 through the channel 164 of the handle 160. Advantageously, the sheath 150 can maintain the catheter body 132, and any lubricious coating disposed thereon, within a sterile environment even during placement of the catheter body 132. The user can then advance the hub 140 towards the handle 160 to advance the catheter body 132 through the urethra and access a bladder. When the eyelet 142 accesses the bladder a fluid can flow proximally through the lumen 134 of the catheter body 132. Advantageously, the handle 160, sheath 150 and hub 140 can contain the catheter body 132 or portions that are to enter the body of the patient within a sterile environment and mitigate direct touch contact. This can prevent the introduction of pathogens or similar catheter associated urinary tract infection (CAUTI) causing agents.

In an embodiment, the user can place the catheter body 132 within the body of the patient prior to coupling the hub 140 with the inlet 116. In an embodiment, the user can couple the hub 140 with the inlet 116 prior to placing the catheter body 132 within the body of the patient. As shown in FIG. 3D, the system 100 is then transitioned to a drainage state and can drain a fluid from the bladder, through the catheter body lumen 134, and into the interior cavity 114 of the body 112. As such, in the drainage state the outer packaging 110 can become a fluid collection container. In an embodiment, once the fluid has be drained from the bladder, a user can remove the catheter body 132 from the urethra by urging the hub 140 distally away from the handle 160. As such, the catheter body 132 is withdrawn through the handle channel 164 and can be contained within the sheath 150.

In an embodiment, once the fluid has been drained, the user can detach the hub 140 from the inlet 116 and can reengage the cap 118 with the inlet 116 creating a fluid tight seal therebetween. Advantageously, the outer packaging 110 can contain and store the fluid within the interior cavity 114 until the fluid and/or packaging 110 can be disposed of discreetly. Since the cap 118 creates a fluid tight seal with in the inlet, the outer packaging 110 containing the fluid can be placed or stored without spilling any fluid. For example, the outer packaging 110 can be placed on a surface, or placed within a pocket or bag until the fluid and/or packaging 110 can be disposed of discreetly. In an embodiment, the sealed packaging 110 including the fluid therein, can be disposed of together. Alternatively, the cap 118 can be removed and the fluid can be emptied from the cavity 114 and, optionally, the body 112 folded or rolled up and disposed of.

In an embodiment, the user can retract the catheter body 132 back within the sheath 150 by urging the hub 140 away from the handle 160. In an embodiment, a distal tip of the handle 160 engage an outlet at a proximal end 148 of the hub 140 in one of a snap-fit, interference fit, press-fit, bayonet-fit, luer-lock, a threadable engagement, or similar suitable engagement. As such, the handle 160, hub 140, and sheath 150 can create a loop containing the catheter body 132, and any fluid therein, within a sealed environment. Advantageously, the looped catheter assembly 130 can also be placed on a surface, or placed within a pocket or bag until the catheter assembly 130 can be disposed of discreetly or stored until suitably cleaned for reuse.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A fluid collection system transitionable between a storage state and a drainage state, the system comprising:
an outer packaging defining an interior cavity and including an inlet in fluid communication with the interior cavity;
a catheter assembly having a distal portion disposed within the interior cavity in the storage state, the catheter assembly comprising:
a catheter body defining a catheter lumen and including an eyelet disposed at the distal tip to provide fluid communication with the catheter lumen;
a hub coupled to a proximal end of the catheter body and defining an outlet in fluid communication with the catheter lumen, the hub engaged with the inlet in the storage state; and
a collapsible sheath extending distally from the hub and extending co-axially over the catheter body.

2. The fluid collection system according to claim 1, wherein the hub, in the storage state, is releasably secured within the inlet in an interference-fit, press-fit, snap-fit, or threaded engagement.

3. The fluid collection system according to claim 1, wherein a proximal end of the hub extends from the inlet when the fluid collection system is in the storage state.

4. The fluid collection system according to claim 1, further including an abutment disposed distally of the outlet and configured to abut against a portion of the inlet to prevent the outlet from engaging the inlet in the storage state.

5. The fluid collection system according to claim 1, wherein the catheter assembly is configured to be removed from the interior cavity and the outlet disposed at a proximal end of the hub is coupled to the inlet to transition the system to the drainage state.

6. The fluid collection system according to claim 1, wherein the hub outlet engages the inlet in one of an interference-fit, press-fit, snap-fit, threaded, bayonet, or luer lock engagement in the drainage state.

7. The fluid collection system according to claim 1, further including a loop coupled to a bottom edge of the outer packaging and configured to engage the inlet in the storage state.

8. The fluid collection system according to claim 1, wherein the catheter assembly is configured to drain a fluid from a bladder of a patient and the outer packaging is configured to collect the fluid within the interior cavity.

9. The fluid collection system according to claim 1, further including a cap configured to releasably engage the inlet and cover one or both of the inlet and the hub outlet.

10. The fluid collection system according to claim 9, wherein the cap is configured to form a fluid tight seal with the inlet when engaged therewith.

11. The fluid collection system according to claim 1, further including a handle coupled to a distal end of the sheath and slidably engaged with the catheter body, the handle configured to guide the distal tip of the catheter body into a urethra and collapse the sheath as the distal tip is advanced distally.

12. The fluid collection system according to claim 11, wherein a distal tip of the handle is configured to engage the outlet of the hub to form the sheath into a loop and contain the catheter body therein.

13. A method of use for a fluid drainage system, comprising:
providing the fluid drainage system in a storage state, comprising:
an outer packaging defining an interior cavity and including an inlet;
a catheter assembly, a distal portion thereof disposed within the interior cavity, the catheter assembly comprising:
a catheter body defining a catheter lumen;
a hub coupled to a proximal end of the catheter body and defining an outlet in fluid communication with the catheter lumen; and
a collapsible sheath extending distally from the hub and extending co-axially over the catheter body;
grasping a proximal end of the hub, a distal portion of the hub releasably secured within the inlet;
removing the catheter assembly from the interior cavity; and
coupling the proximal end of the hub with the inlet to place the catheter lumen in fluid communication with the interior cavity and transition the fluid drainage system to the drainage state.

14. The method according to claim 13, wherein the hub, in the storage state, is releasably secured within the inlet in an interference-fit, press-fit, snap-fit, or threaded engagement.

15. The method according to claim 13, wherein the proximal end of the hub extends from the inlet when the fluid drainage system is in the storage state.

16. The method according to claim 13, further including abutting an abutment, disposed distally of the proximal end of the hub, against a portion of the inlet to prevent the proximal end of the hub from engaging the inlet in the storage state.

17. The method according to claim 13, further including engaging the proximal end of the hub with the inlet in one of an interference-fit, press-fit, snap-fit, threaded, bayonet, or luer lock engagement, in the drainage state.

18. The method according to claim 13, further including coupling a loop, which is attached to a bottom edge of the outer packaging opposite the inlet, with the inlet in the storage state to secure the outer packaging in a folded state.

19. The method according to claim 13, further including draining a fluid from the catheter body into the interior cavity of the outer packaging.

20. The method according to claim 13, further including releasably engaging a cap with the inlet to cover one or both of the inlet and the proximal end of the hub.

21. The method according to claim 20, further including forming a fluid tight seal between the cap and the inlet when the cap is engaged therewith.

22. The method according to claim 13, further including urging a handle proximally towards the hub, to collapse the sheath between the handle and the hub and to advance a distal tip of the catheter body distally of the handle, the handle coupled to a distal end of the sheath.

23. The method according to claim 22, further including sliding the handle distally from the hub to withdraw a portion of the catheter body through the handle and into the sheath and coupling a distal tip of the handle with a proximal end of the hub to form the sheath into a loop and contain the catheter body therein.

24-39. (canceled)
